(19) United States
(12) Patent Application Publication
(10) Pub. No.: US 2008/0004523 A1
(43) Pub. Date: Jan. 3, 2008

(54) Surgical tool guide

(75) Inventor: Vernon Thomas Jensen, Draper, UT (US)

Correspondence Address: MCANDREWS HELD & MALLOY, LTD, 500 WEST MADISON STREET, SUITE 3400, CHICAGO, IL, 60661, US

(73) Assignee: General Electric Company

(21) Appl. No.: 11/477,889

(22) Filed: Jun. 29, 2006

(51) Int. Cl. A61B 5/05

(52) U.S. Cl. 600/424

(57) ABSTRACT

Certain embodiments of the present invention provide n system for orienting a surgical tool with respect to a patient including: a tool guide for facilitating orientation of the surgical tool with respect to the patient, the tool guide including a mounting portion and a tool receiving portion, wherein the tool guide is capable of being integrated with at least a portion of a radiological imaging subsystem including an adjustably moveable mounting structure. In an embodiment, the tool receiving portion is capable of receiving an end-effector. In an embodiment the at least a portion of the radiological imaging subsystem comprises a C-arm. In an embodiment the radiological imaging subsystem comprises a fluoroscopic imaging subsystem. In an embodiment, the end-effector comprises at least one of: an aperture, a cutting device, a drilling device, a clamp, a sleeve, a mounting surface, a ring, a rail, a threaded shaft, a clasp, a bayonet mount, an imaging device, an ultrasound probe, a surgical tool, a catheter, a pin, a screw, a plate, a drill, an awl, and a probe. In an embodiment, the tool guide further comprises an end-effector. In an embodiment, a position of the surgical tool is capable of being adjusted by automatically moving the adjustably movable mounting structure. In an embodiment, the system further comprises at least one position sensing subsystem for ascertaining a position of the surgical tool with respect to the patient.

### BACKGROUND OF THE INVENTION

Embodiments of the present application relate generally to facilitating surgical procedures. Particularly, certain embodiments relate to providing systems, methods, and apparatuses for guiding a tool that may facilitate surgery.

Surgery may have associated risks. It may, therefore, be desirable to both clinicians and patients to reduce both the magnitude and probability of any such surgical risks. One way to reduce risk may be to improve guidance of surgical tools.

A C-arm may be used in radiological imaging subsystems, such as a fluoroscopic imaging system, for example. One trend in radiological imaging subsystems may involve improved positioning sub-systems. For example, in fluoroscopic imaging systems, recent developments have brought about improved precision motorized gantries, and a proliferation of tracking technology for surgical navigation.

Surgical navigation may provide enhanced guidance and visualization, for example. For example, a clinician, such as a surgeon, may wish to place a surgical bone screw to stabilize a fracture site. A tool such as bone screw driver may be used. The clinician may determine tool trajectory from any of a variety of views—for example, multi-planar, or multi-pose image views. The surgeon may then manually maintain the determined trajectory to place the bone-pin, for example. For example, the surgeon may manually maintain a tool(s) as she drills, screws, or otherwise affixes the bone pin to the fracture site. Similarly, clinicians, such as surgeons, may have to manually maintain one or more surgical tools after determining a tool trajectory, for example. Other examples may include therapies that make use of surgical navigation, such as bone biopsies, tissue biopsies, pain management, and/or ablation, for example.

Surgical navigation may also complicate workflow, increase costs, and clutter a surgical environment, for example. One solution to maintaining tool trajectory is to employ robots, for example. Surgical robots may provide relatively rigid and accurate tool guidance for surgery, for example. However, surgical robots may be relatively expensive, and may also take up the space in a potentially crowded surgical environment, for example. It may be, for example, that both a C-arm and a surgical robot may need to occupy the same or an overlapping space. In such a case, it may be necessary to move equipment back and forth during a procedure.

Thus, there is a need to reduce risks associated with surgical procedures. There is a need to provide surgical tool guidance with reduced cost. Additionally, there is a need to provide surgical tool guidance without causing unnecessary crowding in a surgical environment. Moreover, there is a need to provide surgical tool guidance without disrupting workflow in an operating environment.

### BRIEF SUMMARY OF THE INVENTION

Certain embodiments of the present invention provide a system for orienting a surgical tool with respect to a patient including: a tool guide for facilitating orientation of the surgical tool with respect to the patient, the tool guide including a mounting portion and a tool receiving portion, wherein the tool guide is capable of being integrated with at least a portion of a radiological imaging subsystem including an adjustably moveable mounting structure. In an embodiment, the tool receiving portion is capable of receiving an end-effector. In an embodiment the at least a portion of the radiological imaging subsystem comprises a C-arm. In an embodiment the radiological imaging subsystem comprises a fluoroscopic imaging subsystem. In an embodiment, the end-effector comprises at least one of: an aperture, a clamp, a sleeve, a mounting surface such as a slot, a ring, a rail, a threaded shaft, a clasp, a bayonet mount., an imaging device such as an endoscope, an ultrasound probe, a surgical tool or device such as a needle, a catheter, a pin, a screw, a plate, a drill, and awl, a probe, or robot. In an embodiment, the tool guide further comprises an end-effector. In an embodiment, a position of the surgical tool is capable of being adjusted by automatically moving the adjustably movable mounting structure. In an embodiment, the system further comprises at least one position sensing subsystem for ascertaining a position of the surgical tool with respect to the patient. In an embodiment, the adjustably moveable mounting structure is capable of being automatically moved based at least on the position of the surgical tool with respect to the patient. In an embodiment, the at least one sensor comprises at least one of: an image registration subsystem; an imaging subsystem; an optical navigational subsystem; and an electromagnetic navigational subsystem. In an embodiment, the system further comprises an integration mechanism mounted to the adjustably moveable mounting structure for releasably integrating the tool guide to the adjustably moveable mounting structure.

Certain embodiments of the present invention provide a method for orienting a surgical tool with respect to a patient including: integrating a tool guide with at least a portion of a radiological imaging subsystem, the tool guide capable of receiving the surgical tool; and adjusting a position of the at least a portion of the radiological imaging subsystem, wherein a position of the surgical tool is adjusted with respect to the patient. In an embodiment, the method further comprises tracking the position of the surgical tool with respect to the patient to form a tracked position. In an embodiment, the adjusting the position of the adjustably moveable mounting structure is automatically performed based at least in part on the tracked position. In an embodiment, the method further comprises adjusting a position of an end-effector on the tool guide. In an embodiment, the adjusting the position of the adjustably moveable mounting structure is performed according to a planned trajectory of the surgical tool with respect to the patient. In an embodiment, the integrating a tool guide with the adjustably moveable mounting structure is performable with an integration mechanism for releasably integrating the tool guide with the adjustably moveable mounting structure.

Certain embodiments of the present invention provide a method for orienting a surgical tool with respect to a patient including: engaging the surgical tool with a tool guide, the tool guide integrated with an adjustably moveable mounting structure; positioning a position of the surgical tool with respect to the patient; ascertaining a tracked position of the surgical tool with respect to the patient; and determining whether the tracked position of the surgical tool with respect to the patient corresponds to a desired position of the surgical tool with respect to the patient. In an embodiment, the method further comprises adjusting a position of the adjustably moveable mounting structure to cause a corresponding adjustment in the position of the surgical tool with respect to the patient in response to determining whether the tracked position of the surgical tool with respect to the patient corresponds to the desired position of the surgical tool with respect to the patient. In an embodiment, the adjusting the position of the surgical tool is performed automatically. In an embodiment, the desired position of the surgical tool with respect to the patient is based at least in part on a surgical navigation plan.

Certain embodiments of the present invention provide a system for orienting a surgical tool with respect to a patient including: a tool guide for facilitating orientation of the surgical tool with respect to the patient, the tool guide including a mounting portion and a tool receiving portion, wherein the tool guide is capable of being integrated with at least a portion of an image intensifier subsystem including an adjustably moveable mounting structure.

Certain embodiments of the present invention provide a system for orienting a surgical tool with respect to a patient including: a tool guide for facilitating orientation of the surgical tool with respect to the patient, the tool guide including a mounting portion and a tool receiving portion, wherein the tool guide is capable of being integrated with at least a portion of a navigation subsystem including an adjustably moveable mounting structure.

Certain embodiments of the present invention provide a system for orienting a surgical tool with respect to a patient including: a tool guide for facilitating orientation of the surgical tool with respect to the patient, the tool guide including a mounting portion and a tool receiving portion, wherein the tool guide is capable of being integrated with at least a portion of an aiming subsystem including an adjustably moveable mounting structure.

### BRIEF DESCRIPTION OF SEVERAL VIEWS OF THE DRAWINGS

FIG. 1 shows a system for orienting a surgical tool with respect to a patient, in accordance with an embodiment of the present invention.

FIG. 2, shows systems for orienting a surgical tool with respect to a patient, in accordance with an embodiment of the present invention.

FIG. 3 shows a flow chart 300 for a method for orienting a surgical tool with respect to a patient, in accordance with an embodiment of the present invention.

FIG. 4 shows a flow chart 400 for a method for orienting a surgical tool with respect to a patient, in accordance with an embodiment of the present invention.

The foregoing summary, as well as the following detailed description of certain embodiments of the present application, will be better understood when read in conjunction with the appended drawings. For the purpose of illustrating the invention, certain embodiments are shown in the drawings. It should be understood, however, that the present invention is not limited to the arrangements and instrumentality shown in the attached drawings. Further, some figures may be representations of the type of display and/or output associated with methods and systems of the present invention, in accordance with one or more embodiments.

### DETAILED DESCRIPTION OF THE INVENTION

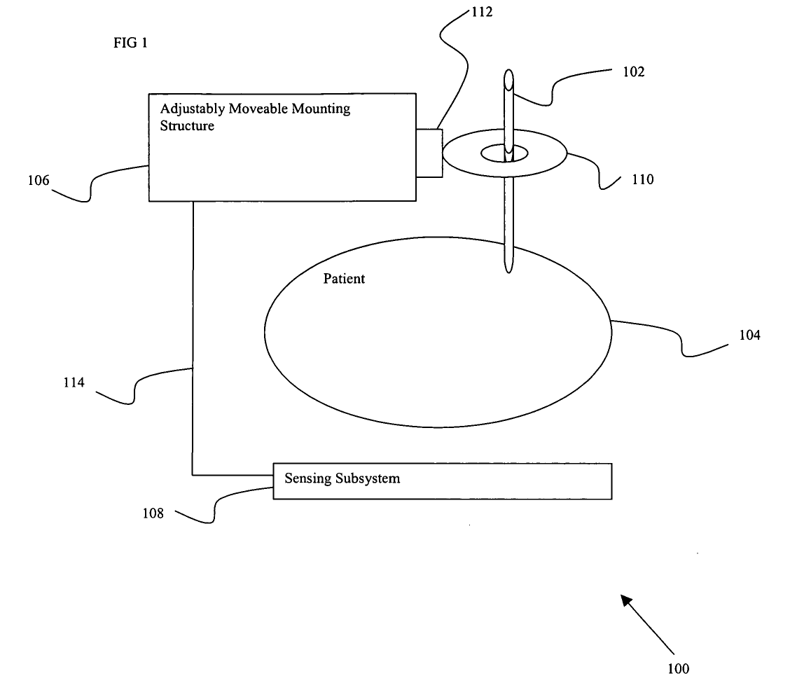

FIG. 1 shows a system 100 for orienting a surgical tool 102 with respect to a patient 104, in accordance with an embodiment of the present invention. The system 100 may include an adjustably moveable mounting structure 106 and a tool guide 110, for example. Optionally, the system 100 may also include a sensing subsystem 108, and/or an attachment mechanism 112, for example.

An adjustably moveable mounting structure 106 may be moveable in any of a number of directions and/or dimensions, such as linearly along one or more Cartesian axes (e.g. x, y, z), and/or radially, for example. The adjustably moveable mounting structure 106 may be moved to adjust the location (e.g. along a coordinate system), and/or may be moved to adjust the spatial orientation of the mounting structure (e.g. angulation of the structure 106 with respect to other objects of interest, such as the patient 104), for example. A position of the moveable structure 106 may include location and/or spatial orientation aspects, for example. The adjustably moveable mounting structure 106 may be adjusted manually and/or automatically, for example. Adjustment may be performable through a lever, a knob, a handle, a wheel, or by an automated device like a solenoid, a servo, or a motor. The mounting structure 106 movement may be controlled by a controller, computer, processor, and/or the like, for example. Movement of the mounting structure 106 may be based at least in part on information received from other components, as will be described below, for example.

The mounting structure 106 may be a portion of any of a variety of equipment available in an operating room, for example. For example, mounting structure 106 may be a portion of a radiological imaging subsystem, a navigation subsystem, or an aiming subsystem. Mounting structure 106 may be, for example, on a C-arm of a radiological imaging subsystem, such as a fluoroscopic imaging subsystem.

The attachment mechanism 112 may be used to integrate the tool guide 110 to the mounting structure 106 in a releasable manner, for example. The attachment mechanism 112 may include latches, screws, clamps, bolts, or other forms of hardware which enable a tool guide 110 to be releasably integrated with the mounting structure 106, for example. The attachment mechanism 112 may form one or more portion, for example. The integration mechanism 112, for example, may have a portion semi-permanently mounted to the mounting structure 106, and another portion for engaging with the mounted portion, for example. The integration mechanism 112 may be capable of releasably integrating with the mounting structure 106 any of a variety of tool guides 110 and/or end-effectors, for example.

The sensing subsystem 108 may optionally be included in system 100, for example. The sensing subsystem 108 may include any of a variety of subsystems, such as an image registration subsystem, an imaging subsystem, an optical navigational subsystem, an electromagnetic navigational subsystem, and/or the like, for example. The sensing subsystem 108 may be separate from a radiological imaging subsystem, or may be incorporated within a radiological imaging subsystem (either partially or completely incorporated), for example. A sensing subsystem 108 may also communicate with other subsystems, such as a radiological imaging subsystem, for example. A sensing subsystem 108 may be able to detect a position of the surgical tool 102 with respect to the patient in one, two, and/or three dimensions, for example. The sensing subsystem 108 may image the surgical tool 102, or otherwise gather information about the location (e.g. in Cartesian coordinates), and/or the orientation of the surgical tool 102, for example.

A sensing subsystem 108 may communicate information, for example, through connection 114 (which may be wireless and/or wired, for example), to convey location and/or orientation information of the surgical tool 102 with respect to the patient. The location and/or orientation information of the surgical tool 102 may be used to control automatically and/or manually the movement of the adjustably moveable mounting structure 106, for example. Thus, sensing subsystem 108 may form a feedback loop in system 100 under certain circumstances, for example.

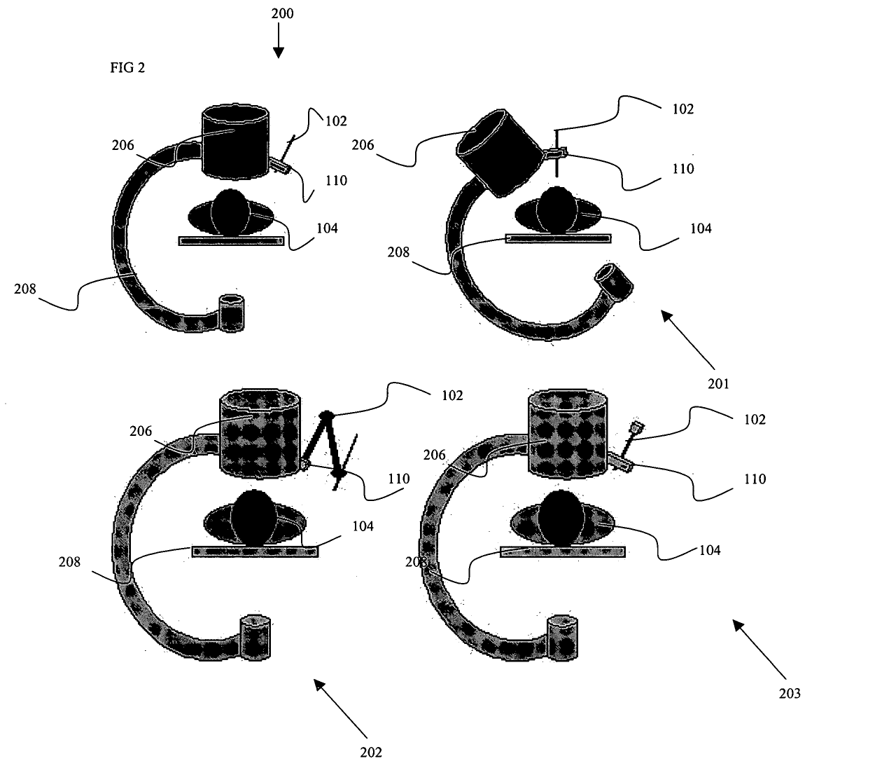

The tool guide 110 may be used to guide, orient, support, or otherwise assist with the positioning of a surgical tool 102 with respect to a patient, for example. The tool guide 110 may interface with a range of surgical tools 102, or different tool guides 110 may be used for different surgical tools 102. Turning for a moment to FIG 2, several different surgical tools 102 are shown in combination with tool guide(s) 110. As discussed, the tool guide 110 may be integrated with a mounting structure 106, either directly, or through an integration mechanism 112, for example. The tool guide 110 may be attachably releasable, or semi-permanently attached or permanently attached to the mounting structure 106, for example.

The tool guide 110 may include an end-effector, and/or may be used for mounting an end-effector, for example. An end-effector may include any portion capable of guiding, orienting, supporting, stabilizing, or otherwise positioning the movement of a surgical tool 102 in one or more dimensions, for example. An end-effector may be as simple as an aperture that restricts the movement of a surgical tool, such as a drill bit, for example. An end-effector may be relatively complex, such as a mini-robot, for example. One example of an end-effector is a Mazor spine robot. Other types of end-effectors may include a cutting or drilling device, a clamp, a sleeve, a mounting surface such as a slot, a ring, a rail, a threaded shaft, a clasp, a bayonet mount, an imaging device such as an endoscope, an ultrasound probe, a surgical tool or device such as a needle, a catheter, a pin, a screw, a plate, a drill, and awl, a probe,

FIG. 2 shows various systems 200-203 for orienting surgical tools 102 with respect to a patient 104, in accordance with an embodiment of the present invention. Systems 200-203 may be similar in many respects to system 100, for example. The mounting structure 206 may be similar to mounting structure 106, for example. As shown in systems 200-203, the mounting structure 206 may form a portion of a C-arm on a radiological imaging subsystem, for example. The movement of a C-arm in a radiological imaging subsystem (e.g. a fluoroscopic imaging system) may cause a corresponding movement of the tool guide 110. Systems 200 and 201 illustrate how movement of the mounting structure 206 may cause movement of the tool guide and corresponding surgical tool with respect to the patient, for example. Systems 202 and 203 illustrate different types of tool guides 110 having or interfacing with different types of end-effectors, for example. For example, system 202 shows a tool guide 110 with an articulating arm, with or without positive feedback. For example, system 203 shows a tool guide 110 with a sensor for determining position and/or orientation relative to a given workspace.

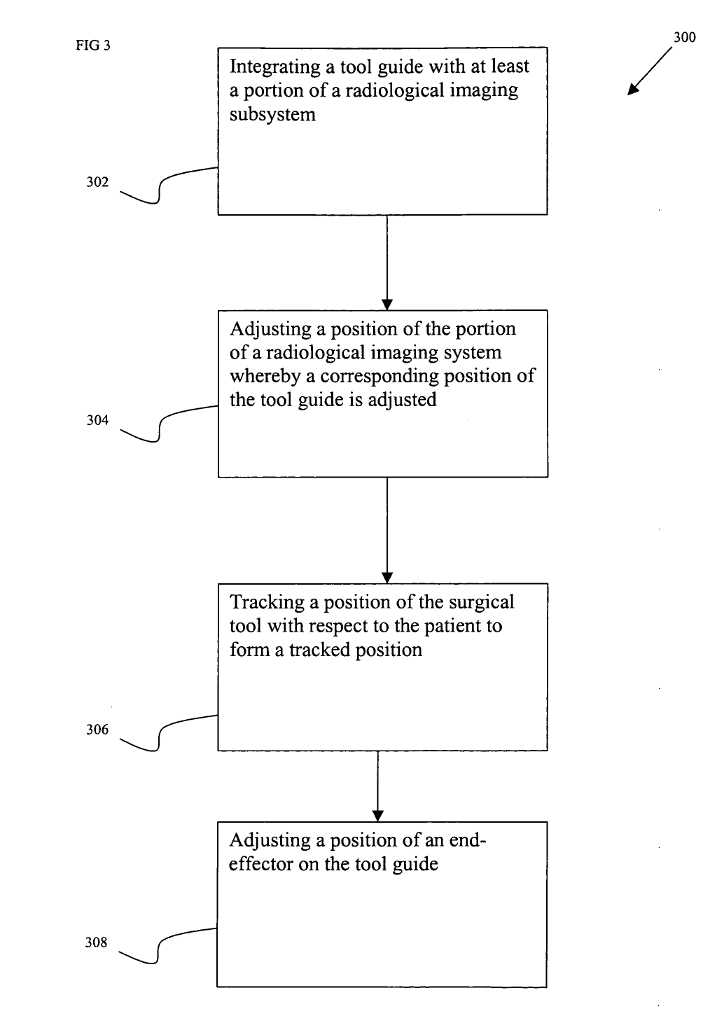

FIG. 3 shows a flow chart 300 for a method for orienting a surgical tool with respect to a patient, in accordance with an embodiment of the present invention. At least a portion of steps of method 300 may be performed in an alternate order and/or substantially/partially simultaneously, for example. For example, step 304 may be performed at the same time as step 306, or step 304 may be performed after step 306. Some steps of method 300 may also be omitted, for example, steps 306 and 308. Method 300 may be performed, in part, by one or more processors, for example.

At step 302 a tool guide may be integrated with at least a portion of a radiological imaging subsystem, for example. A tool guide, such as tool guide 110, may be permanently or semi-permanently integrated with a portion of a radiological imaging subsystem, for example. A tool guide may also be releasably integrated with a radiological imaging subsystem through an integration mechanism, such as integration mechanism 112. A tool guide may be integrated into a portion of a radiological imaging subsystem, such as an adjustably moveable mounting structure 106, for example. A tool guide may be integrated, for example, onto a portion of the C-arm of a radiological imaging subsystem, for example. Different styles of tool guides may be releasably, permanently, or semi-permanently integrated with the radiological imaging subsystem, for example.

At step 304 a position of a radiological imaging subsystem may be adjusted, whereby a corresponding position of the tool guide is adjusted, for example. A particular portion of the radiological imaging subsystem position may be adjusted, or the entire imaging system position may be adjusted, for example. The radiological imaging subsystem position may be adjusted automatically and/or manually, for example. For example, a portion of the radiological imaging subsystem may include a C-arm, and the C-arm may be adjustably moveable, either automatically, or manually, for example. The position may be adjusted in response to other steps, such as step 306 and/or step 308, for example. The position may be adjusted by a remote controller, or locally through the radiological imaging subsystem, for example.

At step 306 a position of the surgical tool with respect to the patient may be tracked to form a tracked position. The position of the surgical tool may be tracked with a variety of tracking subsystems such as an imaging subsystem (e.g. the radiological imaging system discussed above), an image registration subsystem, an optical navigation subsystem, an electromagnetic navigation subsystem, and/or the like. The position of the surgical tool may be tracked in one or more dimensions, for example, with respect to the patient. The position may be tracked with respect to other objects such as fiducials, and/or with respect to virtual objects, such as surgical plans, for example. The tracked position information may be tracked substantially in real-time, for example. The tracked position may be communicated to one or more other components, such as a subsystem controlling the movement of the radiological imaging system, for example. The tracked position may include both location information (e.g. Cartesian coordinates) and/or orientation information of the surgical tool, for example.

At step 308 a position of an end-effector on the tool guide may be adjusted. For example, an end-effector may comprise a robot, such as a Mazor spine robot. The end-effector may thus be capable itself of articulation and/or otherwise adjusting the location and/or orientation of the surgical tool, for example. The position of the end-effector may be adjusted in coordination with the adjustment performed in step 304, or may be independently adjusted, for example.

As an illustrative example, a method described in association with FIG. 3 may be performed in the following manner. A particular tool guide is designed for restricting motion of a drill bit. The drill bit is used for drilling into a patient's bone. At step 302, the “drill bit” tool guide is integrated with a C-arm on a radiological imaging subsystem through an integration mechanism (see FIGS. 1, 2). The integration mechanism allows for tool guides to be releasably integratable with the C-arm. At step 304, the C-arm of the imaging subsystem is automatically adjusted in accordance with a surgical plan. The surgical plan includes trajectories for the path of the drill bit into the patient's bone, for example. The movement of the C-arm causes a corresponding movement of the tool guide and surgical tool, such that the position of the drill bit becomes aligned with the surgical plan. The adjustment in step 304 may be assisted with step 306, in which the position of the drill bit is tracked with respect to the patient. The drill bit's coordinates are determined with respect to the patient's bone, substantially in real-time, through a surgical navigation subsystem, such as an electromagnetic navigation subsystem. The tracked position of the drill bit is compared with the surgical plan to determine if the drill bit is following the planned trajectory. When the drill bit position strays from the planned trajectory, the radiological imaging subsystem C-arm is automatically moved to compensate for any discrepancy. Finally, at step 308, an end-effector, such as a Mazor spine robot may also be provided to further adjust the position of the surgical tool.

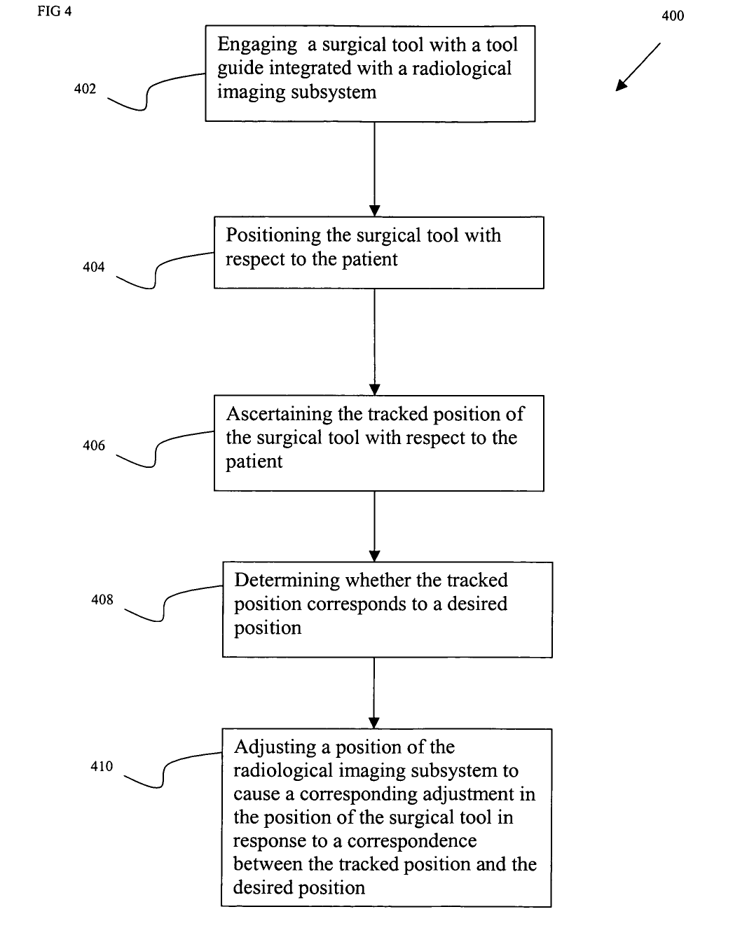

FIG. 4 shows a flow chart 400 for a method for orienting a surgical tool with respect to a patient, in accordance with an embodiment of the present invention. At least a portion of steps of method 400 may be performed in an alternate order and/or substantially/partially simultaneously, for example. For example, step 404 may be performed at the same time as step 406, or step 404 may be performed after step 406. Some steps of method 400 may also be omitted, for example, step 410. Method 400 may be performed, in part, by one or more processors, for example.

At step 402 a surgical tool (e.g. surgical tool 102) may be engaged with a tool guide (e.g. tool guide 110) integrated with a radiological imaging subsystem. For example, a surgical tool (e.g. drill, drill bit, ablation tool, scope, etc.) may be engaged with the tool guide. The tool guide may have various adapters, designs, and/or end-effectors to accommodate various tools, for example. Alternatively, different types of tool guides may be designed to engage various surgical tools, for example. The tool guide itself may be integrated with a radiological imaging subsystem, either releasably, permanently, or semi-permanently, as discussed above, for example. The surgical tool may be engaged with the tool guide such that the surgical tool is moveable and/or adjustable, for example. As an example, a tool guide may engage a drill bit, such that the bit is moveable along the bit's main axis, and rotatable around the main axis. However, the tool guide may substantially restrict motion of the bit along other dimensions, for example. Alternatively, the surgical tool may be engaged so that it is substantially immobile, for example. As an example, a mini-robot may substantially immobilize the surgical tool by clamping the surgical tool. The mini-robot may itself be capable of articulation, but the tool by itself may be substantially immobilized with respect to the clamp, for example.

At step 404 the surgical tool may be positioned with respect to the patient (e.g. patient 104). The surgical tool may be positioned by adjusting the tool with respect to the tool guide. For example, a drill bit may be adjusted along its main axis so that it is in a position with respect to the patient. A surgical tool may be positioned with respect to the patient, and then substantially immobilized with respect to engagement mechanisms on the tool guide, for example. A surgical tool may be positioned with respect to the patient based on clinical needs, a surgical plan, and/or the like, for example. A surgical tool may be positioned with respect to the patient manually and/or automatically, for example.

At step 406 the tracked position of the surgical tool with respect to the patient may be ascertained. The tracked position of the surgical tool may be ascertained through any of a variety of subsystems, such as the following; imaging subsystem, optical navigation subsystem, electromagnetic navigation subsystem, image registration subsystem, and/or the like, for example. An electromagnetic navigation subsystem may track the position of the tool with electromagnetic information; for example. Examples of electromagnetic navigation subsystems include passive sensing systems and active sensing systems. For example, the tool itself may emit an electromagnetic signal, or the tool may have an antenna pattern recognizable to sensors located outside the patient, for example. The tracked position may include location information (e.g. Cartesian coordinates) in one or more dimensions, for example. The tracked position may also include orientation information of the surgical tool with respect to the patient, or other references such as fiducials, for example.

At step 408 whether the tracked position corresponds to a desired position may be determined. A desired position may include location information in one or more dimensions for example. A desired position may also include orientation information (orientation, for example, of the surgical tool with respect to the patient), for example. A desired position may be determinable based on clinical interest, for example. A desired position may be determinable based on a surgical plan, for example. A surgical plan may include tool trajectory information, for example. A surgical plan may be developed prior to surgery, or during surgery, for example.

At step 410 a position of the radiological imaging subsystem may be adjusted to cause a corresponding adjustment in the position of the surgical tool in response to a correspondence between the tracked position and the desired position. The adjustment of step 410 may be performed automatically and/or manually, for example. The adjustment may be performed by adjusting the position, for example, a C-arm on a radiological imaging subsystem, for example, as shown in FIG. 2.

As an illustrative example, a method described in association with FIG. 4 may be performed in the following manner. A surgical tool in this example is an ablation tool, and a radiological imaging subsystem is a fluoroscopic imaging subsystem having a C-arm. At step 402, The ablation tool is engaged with a tool guide that is integrated with the fluoroscopic imaging subsystem. The tool guide restrains the motion of the ablation tool in every direction except along the main axis of the ablation tool. The ablation tool is engaged with the tool guide by inserting the tool through an aperture on the tool guide. The ablation tool is then, at step 404, positioned by a clinician who follows a surgical plan. The ablation tool is positioned, then, in accordance with the surgical plan within a tumor to be ablated. Then, at step 406, a surgical navigation system determines the position of the ablation tool (e.g. location, and orientation). For example, an electromagnetic navigation subsystem may function as a surgical navigation system by imaging the tool with respect to the patient to determine position. At step 408, it is determined whether the tracked position of the ablation tool corresponds to the surgical plan. Then, at step 410, when the ablation tool does not correspond to the desired position according to the surgical plan, the position of the C-arm is adjusted an appropriate amount to realign the ablation tool with the intended tool trajectory.

Thus, embodiments of the present application provide reduced risks associated with surgical procedures. embodiments of the present application provide surgical tool guidance with reduced cost. Additionally, embodiments of the present application provide surgical tool guidance without causing unnecessary crowding in a surgical environment. Moreover, embodiments of the present application provide surgical tool guidance without disrupting workflow in an operating environment.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. For example, features may be implemented with software, hardware, or a mix thereof. Therefore, it is intended that the invention not be limited to the particular embodiment disclosed, but that the invention will include all embodiments falling within the scope of the appended claims.

1. A system for orienting a surgical tool with respect to a patient comprising:
a tool guide for facilitating orientation of the surgical tool with respect to the patient, said tool guide comprising a mounting portion and a tool receiving portion,
wherein said tool guide is capable of being integrated with at least a portion of a radiological imaging subsystem comprising an adjustably moveable mounting structure.

2. The system of claim 1, wherein said tool receiving portion is capable of receiving an end-effector.

3. The system of claim 1, wherein said at least a portion of said radiological imaging subsystem comprises a C-arm.

4. The system of claim 1, wherein said radiological imaging subsystem comprises a fluoroscopic imaging subsystem.

5. The system of claim 2, wherein said end-effector comprises at least one of: an aperture, a cutting device, a drilling device, a clamp, a sleeve, a mounting surface, a ring, a rail, a threaded shaft, a clasp, a bayonet mount, an imaging device, an ultrasound probe, a surgical tool, a catheter, a pin, a screw, a plate, a drill, an awl, and a probe.

6. The system of claim 1, wherein said tool guide further comprises an end-effector.

7. The system of claim 1, wherein a position of the surgical tool is capable of being adjusted by automatically moving said adjustably movable mounting structure.

8. The system of claim 1 further comprising at least one position sensing subsystem for ascertaining a position of the surgical tool with respect to the patient.

9. The system of claim 8, wherein said adjustably moveable mounting structure is capable of being automatically moved based at least on said position of the surgical tool with respect to the patient.

10. The system of claim 1, wherein said at least one sensor comprises at least one of: an image registration subsystem; an imaging subsystem; an optical navigational subsystem; and an electromagnetic navigational subsystem.

11. The system of claim 1 further comprising an integration mechanism mounted to said adjustably moveable mounting structure for releasably integrating said tool guide to said adjustably moveable mounting structure.

12. A method for orienting a surgical tool with respect to a patient comprising:
integrating a tool guide with at least a portion of a radiological imaging subsystem, said tool guide capable of receiving the surgical tool; and
adjusting a position of said at least a portion of said radiological imaging subsystem, wherein a position of the surgical tool is adjusted with respect to the patient.

13. The method of claim 12 further comprising tracking said position of the surgical tool with respect to the patient to form a tracked position.

14. The method of claim 13, wherein said adjusting said position of said adjustably moveable mounting structure is automatically performed based at least in part on said tracked position.

15. The method of claim 12 further comprising adjusting a position of an end-effector on said tool guide.

16. The method of claim 12, wherein said adjusting said position of said adjustably moveable mounting structure is performed according to a planned trajectory of the surgical tool with respect to the patient.

17. The method of claim 12, wherein said integrating a tool guide with said adjustably moveable mounting structure is performable with an integration mechanism for releasably integrating said tool guide with said adjustably moveable mounting structure.

18. A method for orienting a surgical tool with respect to a patient comprising:
engaging the surgical tool with a tool guide, said tool guide integrated with an adjustably moveable mounting structure;
positioning a position of the surgical tool with respect to the patient;
ascertaining a tracked position of the surgical tool with respect to the patient; and
determining whether said tracked position of the surgical tool with respect to the patient corresponds to a desired position of the surgical tool with respect to the patient.

19. The method of claim 18 further comprising adjusting a position of said adjustably moveable mounting structure to cause a corresponding adjustment in said position of the surgical tool with respect to the patient in response to determining whether said tracked position of the surgical tool with respect to the patient corresponds to said desired position of the surgical tool with respect to the patient.

20. The method of claim 19, wherein said adjusting said position of the surgical tool is performed automatically.

21. The method of claim 18, wherein said desired position of the surgical tool with respect to the patient is based at least in part on a surgical navigation plan.

22. A system for orienting a surgical tool with respect to a patient comprising:
a tool guide for facilitating orientation of the surgical tool with respect to the patient, said tool guide comprising a mounting portion and a tool receiving portion,
wherein said tool guide is capable of being integrated with at least a portion of an image intensifier subsystem comprising an adjustably moveable mounting structure.

23. A system for orienting a surgical tool with respect to a patient comprising:
a tool guide for facilitating orientation of the surgical tool with respect to the patient, said tool guide comprising a mounting portion and a tool receiving portion,
wherein said tool guide is capable of being integrated with at least a portion of a navigation subsystem comprising an adjustably moveable mounting structure.

24. A system for orienting a surgical tool with respect to a patient comprising:
a tool guide for facilitating orientation of the surgical tool with respect to the patient, said tool guide comprising a mounting portion and a tool receiving portion,
wherein said tool guide is capable of being integrated with at least a portion of an aiming subsystem comprising an adjustably moveable mounting structure.
